(19) United States
(12) Patent Application Publication
(10) Pub. No.: US 2016/0157874 A1
(43) Pub. Date: Jun. 9, 2016

(54) DEVICE FOR ASSISTING INFANT LATCH DURING AND AFTER BREASTFEEDING

(71) Applicant: Fiddniel Mark GUILARTE, Riverview, FL (US)

(72) Inventor: Fiddniel Mark GUILARTE, Riverview, FL (US)

(21) Appl. No.: 14/958,484

(22) Filed: Dec. 3, 2015

Related U.S. Application Data
(60) Provisional application No. 62/086,904, filed on Dec. 3, 2014.

(51) Int. Cl. A61B 17/24

(52) U.S. Cl. CPC A61B 17/24

(57) ABSTRACT

A latch pick assembly providing a pick portion dimensioned and adapted to establish, adjust and break the latch of a breastfeeding infant is provided. The pick portion provides opposing front and rear surfaces that taper toward each other as they terminate at a distal forward edge. The forward edge provides a plurality of protrusions which share the longitudinal and transverse concavity of the front surface and corresponding longitudinal and transverse convexity of the rear surface. The forward edge enables the gentle urging of an infant's lips into positions for a proper latch, and the adjusting and breaking of the latch during breastfeeding.

### CROSS-REFERENCE TO RELATED APPLICATION

This application claims the benefit of priority of U.S. provisional application No. 62/086,904, filed 3 Dec. 2014, the contents of which are herein incorporated by reference.

### BACKGROUND OF THE INVENTION

The present invention relates to breastfeeding and, more particularly, to a device for assisting an infant to latch during breastfeeding.

Currently, women can endure difficulties when breastfeeding their newborns. Difficulties include (1) the effects of improper latching of the nipple that creates soreness, scabbing, and lacerations that tend to lead to infections, (2) maintaining the discipline to continue breastfeeding given the many challenges women face, and (3) the bacteria presented to newborns during current breastfeeding techniques that rely on the mother putting their finger in their infant's mouth to assist in securing a latch.

There are no products currently out there for correcting an infant's latch upon nursing or breastfeeding. Currently, women are using their finger to aid in creating a proper latch for breastfeeding, thus introducing risks of bacteria and “nipple confusion” for the infant. If the mother uses latex gloves to cover her finger, this process is impractical and time consuming. In reference to pacifiers and latex nipples; these items deter both babies and mothers from breastfeeding. It is the complete opposite of promoting breastfeeding and improving the infant's immune system through needed nutrients upon birth.

As can be seen, there is a need for a device for assisting an infant to latch during breastfeeding.

### SUMMARY OF THE INVENTION

In one aspect of the present invention,

In another aspect of the present invention,

These and other features, aspects and advantages of the present invention will become better understood with reference to the following drawings, description and claims.

### BRIEF DESCRIPTION OF THE DRAWINGS

FIG. 1 is a perspective view of an exemplary embodiment of the present invention, shown in use;

FIG. 2 is a front perspective view of an exemplary embodiment of the present invention;

FIG. 3 is a rear perspective view of an exemplary embodiment of the present invention;

FIG. 4 is an exploded view of an exemplary embodiment of the present invention;

FIG. 5 is a section view of an exemplary embodiment of the present invention, taken along line 5-5 of FIG. 2;

FIG. 6 is a section view of an exemplary embodiment of the present invention, illustrating the removal of a latch pick assembly from a housing assembly; and

FIG. 7 is a perspective view of an exemplary embodiment of the present invention, shown in use.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a latch pick assembly providing a pick portion dimensioned and adapted to establish, adjust and break the latch of a breastfeeding infant. The pick portion provides opposing front and rear surfaces that taper toward each other as they terminate at a distal forward edge. The forward edge provides a plurality of protrusions which share the longitudinal and transverse concavity of the front surface and corresponding longitudinal and transverse convexity of the rear surface. The forward edge enables the gentle urging of an infant's lips into positions for a proper latch, and the adjusting and breaking of the latch during breastfeeding.

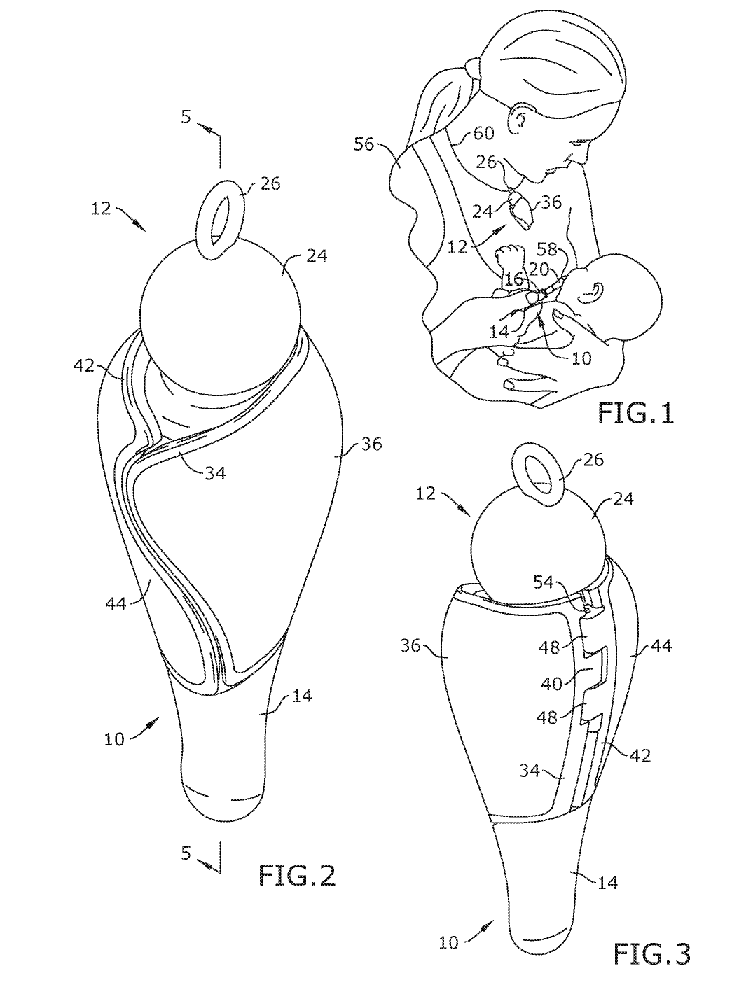

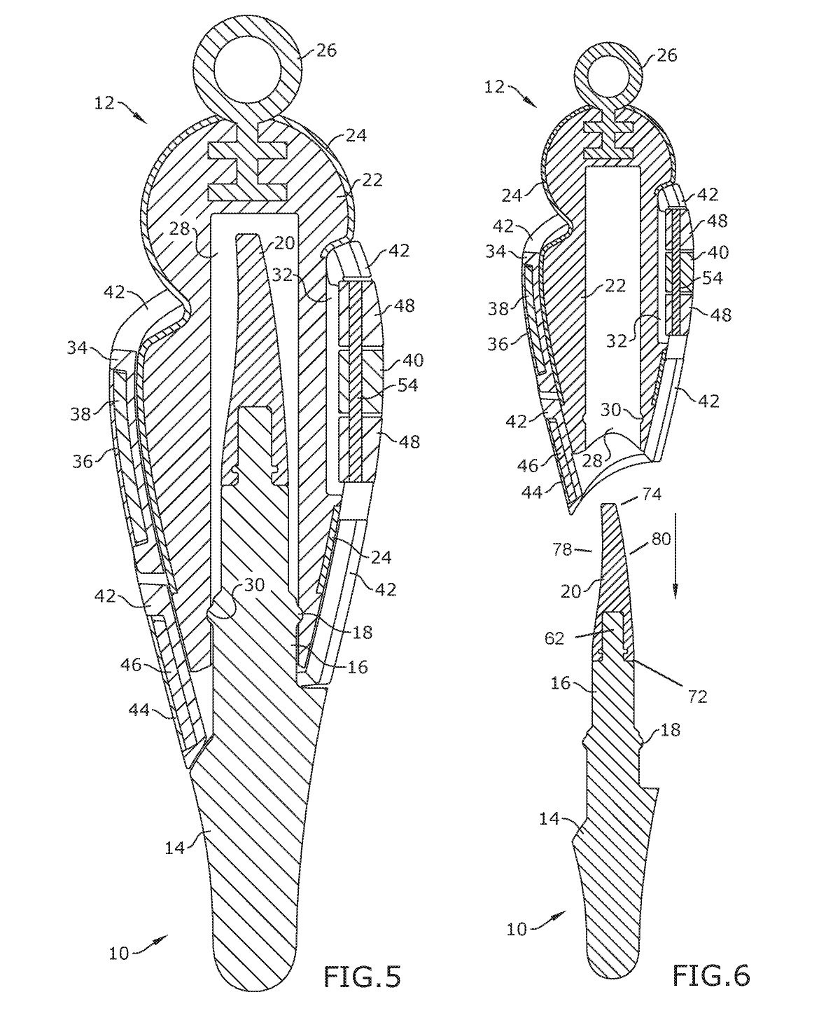

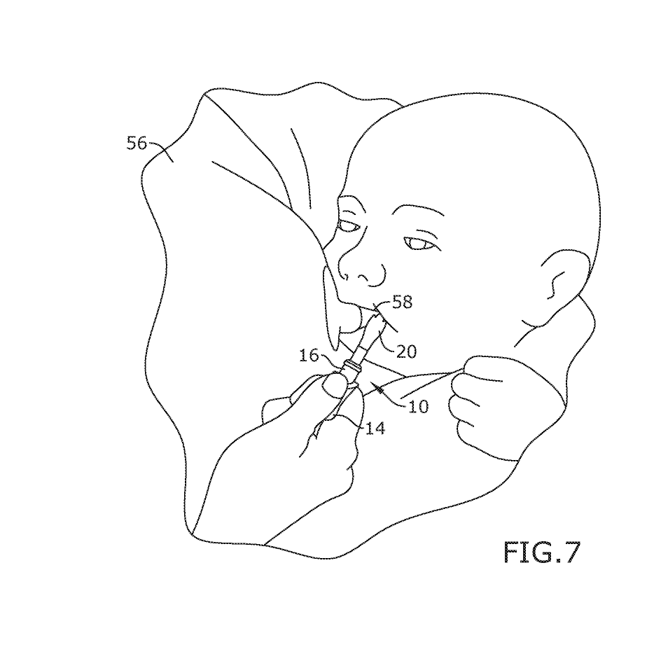

Referring to FIGS. 1 through 7, the present invention may include a latch pick assembly 10 and a housing assembly 12. The latch pick assembly 10 may include a handle portion 14, an arm portion 16 and a pick portion 20. The arm portion 16 may extend from a handle end to a pick end 62. The handle portion 14 may be joined to the handle end of the arm portion 16. The pick portion 20 may removably attach to the pick end 62 of the arm portion 16. The handle portion 14 may be dimensioned and adapted to be manually engaged so that a user 56 may manipulate the pick portion 20. The pick portion 20 may be made of BPA-free, dye-free material and the like. The remaining portions 14 and 16 may be made from any suitable resilient material.

The pick portion 20 may extend along a longitudinal axis from a first end 72 to a second end 74, wherein the first end 72 removably engages the pick end 62, in certain embodiments by forming a cavity to snugly receive the pick end 62. The pick portion 20 may taper from a first thickness to a second thickness as it extends to the second end 74, wherein the second thickness is less than the first thickness.

The pick portion 20 may provide an edge surface 76 that interconnects a front surface 78 and a rear surface 80, wherein the surfaces 76, 78 and 80 define peripheries of the pick portion 20. The surfaces 76, 78 and 80 may be rounded where they interface. The edge surface 76 may be generally analogous with the varying thickness of the pick portion 20, wherein at the first end 72 the edge surface 76 may be generally defined by the first thickness. The front surface 78 may be longitudinally and transversely concave. The rear surface 80 may be longitudinally and transversely convex.

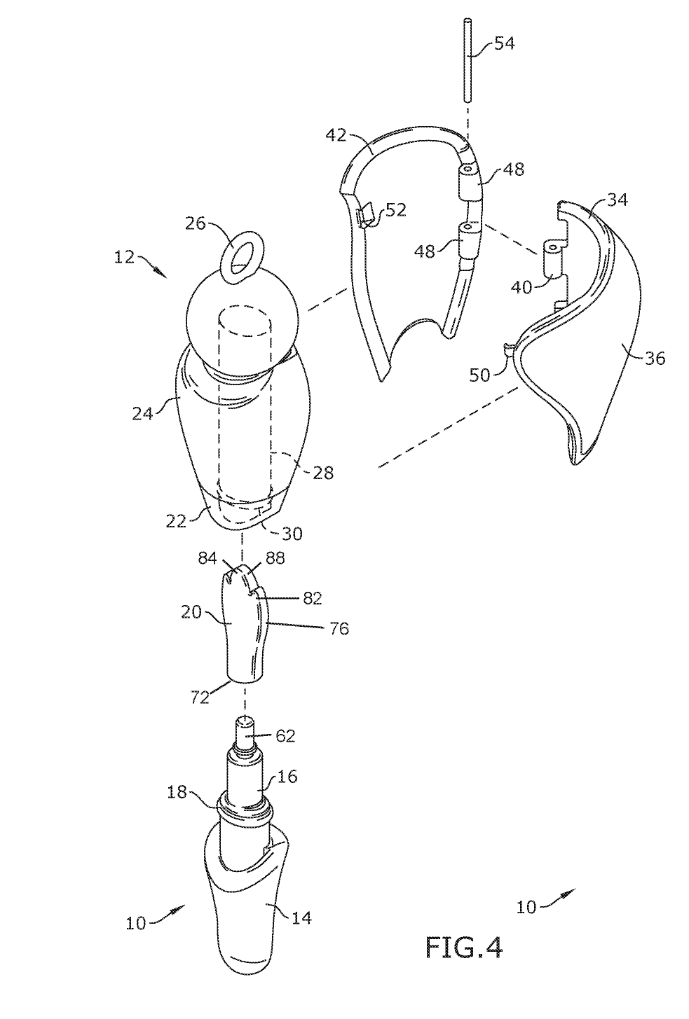

Near the second end 74, the surfaces 76, 78 and 80 may be defined by two spaced about side protrusions 82 and a middle protrusion 84 disposed between the two side protrusions 82, as illustrated in FIG. 4. A distance from a distal end of the middle protrusion 84 to the first end 72 may be greater that a distance from a distal end of either side protrusion 82 to the first end 72. The tapering protrusions 82 and 84 may form a forward edge 88.

In certain embodiments the longitudinal distance from the distal end of the middle protrusion 84 to the first end 72 may be approximately one half inch. The transverse length of the pick portion 20 may be approximately one third of an inch (the transverse length may be defined from the opposing outer surface edges 76 of the two spaced side protrusions 82). The displacement of the rear surface 80 as it extends from the first end 72 to the second end 74/forward edge 88 along a transversely convex path may be approximately 0.3 inches measured along a transverse plane relative to the longitudinal axis. The longitudinal distance from the distal end of the middle protrusion 84 to the pick end 62 of the handle portion 14 may be approximately 1.1 to 1.2 inches in length.

The thickness (edge surface 76) of the pick portion 20 is dimensioned and adapted to not mimic the thickness of a human nipple so that the infant does not attempt to confusingly, instinctively latch onto the pick portion 20 or any of its protrusions 82, 84.

The resulting curvature of the pick portion 20 may be dimensioned and adapted to engage the lips of an infant so as to manipulate the mouth 58 to create, adjust, and break the infant's latch during breastfeeding without the use of their finger. The forward edge 88 may be inserted into the mouth 58 up to at least the middle protrusion 84. The pick portion 20 can be used in many scenarios: (1) to flip the infant's upper and lower lip into the flange position for optimal suction, (2) break a infants latch without an abrupt motion reducing chances of damage to both mother and baby, (3) used to treat lip-tied or tongue-tied babies while nursing to establish the flange position, and (4) post-surgery stretching during the healing process. The manipulation of the mouth 58 via the forward edge 88 and the protrusions 82, 84 may allow the user to tilt the pick portion 20 so as to flip the upper and lower lips easily without breaking the suction by not creating a gap between the infant lips and nipple. The middle protrusion 84 and at least one side protrusion 82 may be placed in the mouth 58 so that the rear surface 80 abuts the upper lip. To then create a proper latch, the user 56 may urge the upper lip to open up more into a flanged position, whereby the difference in lengths of the middle and the side protrusion aids the user 56 in gently flipping the upper lip as the shorter side protrusion 82 will engage a more forward portion of the lip, thus facilitating an “unfurling” motion as compared to a mere lifting motion. The unfurling motion tends to enable the lip to engage more of the area around the nipple, facilitating a more secure latch. And with a subtle flip of the wrist the user 56 may similarly urge the bottom lip down using the front surface 78 and protrusions 82, 84.

Subsequent use, the pick portion 20 may be removably engaged by the housing assembly 12 so as to be hidden from view of the infant and free from external elements.

The housing assembly 12 may form a general pendant shaped lower and upper body 22, 24. The body 22, 24 may form a body cavity 28 for slidably receiving the arm and pick portions 16 and 20. The body cavity 28 may form a groove 30. The arm portion 16 may provide a retaining ring 18 that snugly engages the groove 30 when the pick portion is secured within the body cavity 28.

The housing assembly 12 may include a first and second blanket leaf 34, 42 that encases a portion of the lower and upper body 22, 24, which may form a hinge notch 32 for facilitating the encasement. The first and second blanket leaf 34, 42 may provide a first and second leaf inlay 36, 44, a first and second inlay backer 38, 46, and a first and second hinge knuckle 40, 48, respectively, for facilitating a pivotal connection for encasing the portion of the lower and upper body 22, 24, whereby a hinge pin 54 operably engages the knuckles 40, 48 for such a connection, and whereby complementary tab 50 and slot 52 are provided on the respective leafs 34, 42 for securing said encasement.

A ring 26 may be disposed on the upper body 24 so that the housing assembly 12 may then be attached to a necklace 60 or the like to portably store the latch pick assembly 10. To use the latch pick assembly 10 again, the user 56 may disengage the latch pick assembly 10 from the body cavity 28, exposing the pick portion 20 for use.

It should be understood, of course, that the foregoing relates to exemplary embodiments of the invention and that modifications may be made without departing from the spirit and scope of the invention as set forth in the following claims.

What is claimed is:

1. A latch pick assembly dimensioned and adapted to manipulate lips of an infant for securing, adjusting and breaking a proper breastfeed latch, comprising:
a pick portion extending along a longitudinal axis from a first end to a forward edge, the pick portion having a front surface and an opposing rear surface that taper toward each other as the pick portion extends to the forward edge; and
a plurality of protrusions formed along the forward edge,
wherein the front surface is transversely concave.

2. The latch pick assembly of claim 1, wherein the rear surface is transversely convex.

3. The latch pick assembly of claim 2, wherein the plurality of protrusions comprise:
two spaced side protrusions formed along the forward edge; and
a middle protrusion formed along the forward edge so as to be disposed between the two spaced side protrusions.

4. The latch pick assembly of claim 3, wherein a distance from a distal end of the middle protrusion to the first end is greater than a distance from a distal end of one of the side protrusions to the first end.

5. The latch pick assembly of claim 4, wherein the front surface is longitudinally concave.

6. The latch pick assembly of claim 5, further comprising a handle portion removably attached to the first end.

7. The latch pick assembly of claim 6, further comprising an arm portion interconnecting the handle portion the first end.

8. The latch pick assembly of claim 7, further comprising a housing assembly forming a body cavity dimensioned and adapted to removably house at least the pick portion.

9. The latch pick assembly of claim 4, wherein a transverse length of the pick portion is approximately one third of an inch.

10. The latch pick assembly of claim 9, wherein a transverse displacement of the rear surface is approximately 0.3 inches as the rear surface extends from the first end to the forward edge.
